(19) United States
(12) Patent Application Publication
(10) Pub. No.: US 2007/0221231 A1
(43) Pub. Date: Sep. 27, 2007

(54) METHOD AND APPARATUS FOR TREATMENT OF SNORING AND SLEEP APNEA

(76) Inventor: John A. Macken, Santa Rosa, CA (US)

Correspondence Address: STAINBROOK & STAINBROOK, LLP, 412 AVIATION BOULEVARD, SUITE H, SANTA ROSA, CA, 95403, US

(21) Appl. No.: 11/761,337

(22) Filed: Jun. 11, 2007

Related U.S. Application Data
(63) Continuation-in-part of application No. 11/102,314, filed on Apr. 8, 2005.
(63) Continuation-in-part of application No. 10/866,058, filed on Jun. 10, 2004, now abandoned.

(51) Int. Cl. A61F 5/56

(52) U.S. Cl. 128/848

(57) ABSTRACT

An elongated anti-snoring device has adhesive areas at each end of the device and a central region of the anti-snoring device is designed to exert pressure. In use, the adhesive areas exert a pulling force on two areas of the neck causing these areas of the neck to expand outward from their normal position. The central portion of the anti-snoring device presses on a specific part of the neck to generate the pulling force. The forces on the neck open a blockage in the throat of a sleeping person, thereby reducing both snoring and sleep apnea.

### CROSS REFERENCES TO RELATED APPLICATIONS

The present application is a continuation-in-part of U.S. Utility patent application Ser. No. 11/102,314, filed Apr. 4, 2005 (Apr. 8, 2005), which is a continuation-in-part of U.S. Utility patent application Ser. No. 10/866,058, filed Jun. 10, 2004 (Jun. 10, 2004).

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

1. Field of the Invention

The present invention relates generally to health and medical devices, and more particularly to an improved method and apparatus for the treatment of snoring and sleep apnea.

2. Discussion of Related Art including information disclosed under 37 CFR 61.97, 1.98

The fundamental cause of snoring is a restriction or blockage of the air passage in the throat. This occurs when muscles relax during sleep allowing soft tissue in the back of the throat to sag. This soft tissue then blocks the flow of air needed for breathing. Most people that snore overcome this blockage by breathing harder, and the increased air pressure partially opens up the air passage. The snoring sound is the restricted airflow vibrating the soft tissue. Besides the obvious noise, the snoring person also has laborious breathing that decreases the quality of sleep. In extreme cases of air passage blockage, there is a medical condition called obstructive sleep apnea where the quality of sleep is severely degraded.

There are hundreds of patents relating to anti-snoring devices. Almost all the mechanical devices that have been developed simply do not treat the fundamental cause of snoring because they do not open up the blocked air passage in the throat. There are two classes of mechanical anti-snoring devices that actually open up this blocked air passage. The first class opens the blocked air passage in the throat by forcing the lower jaw to jut forward to an unnatural position. However, these devices that force the lower jaw forward are very uncomfortable and can cause alignment problems with the teeth.

The second class of anti-snoring devices that opens the blocked air passage in the throat utilizes an air mask, a hose and a low-pressure air compressor to inflate the lungs and air passages to a pressure typically between 70 and 120 Pascal above atmospheric pressure (a pressure between about 7 mm and 12 mm of water). This class of device is usually called a CPAP machine (Continuous Positive Airways Pressure machine). These machines are expensive, uncomfortable and restrictive. About half of the people that start using CPAP machines to treat sleep apnea cannot tolerate them and discontinue their use.

All the known prior art anti-snoring devices that utilize an adhesive are intended to be worn somewhere on the face. These devices open the nose, distort the cheek or cover the mouth. None of these prior art adhesive devices stop snoring by expanding a portion of the neck to open up the blocked air passage in the throat.

For example, the following patents utilize an adhesive device to cover the mouth: French Patent No. 78 08083 to Blander; German Patent No. DE 3837277 to Obermeler; and U.S. Pat. No. 4,817,636 to Woods.

The following patents utilize an adhesive or suction device on the nose: U.S. Pat. No. 1,292,083 to Sawyer; U.S. Pat. No. 6,631,714 to Von Duyke; U.S. Pat. No. Re. 35,408 to Petruson; and U.S. Pat. No. 3,594,813 to Sanderson.

The following patents utilize an adhesive device on the cheek: U.S. Pat. No. 3,782,372 to Carlton; and U.S. Pat. No. 5,752,524 to Corcoran.

All the known prior art anti-snoring devices that utilize an implant utilize an implant in the soft palate to alter the dynamic response and reduce snoring. No prior art anti-snoring patents use an implant to expand an external part of the neck. Typical soft palate implant patents are: U.S. Pat. Nos. 6,848,447 6,634,362 6,601,585 6,578,580 6,523,543 to Conrad and U.S. Pat. Nos. 6,626,181 6,601,584 6,523,542 to Knudson.

U.S. Pat. No. 5,592,938 to Scarberry describes a body part enclosure comprising a sheath or rap that can be made rigid by the application of a vacuum. One of the applications briefly mentioned for this device is a sheath to create a partial vacuum on the neck as a treatment for sleep apnea. The vacuum is intended to draw out tissue and expand the airway during sleep to relieve airway obstruction. No specific design is given for a device that would treat sleep apnea.

The Scarberry prior art requires that the vacuum device be attached to a vacuum pump to compensate for minor leaks. Also, movement of a sleeping person can break the vacuum seal and cause a major leak. There will also be a strong pushing force exerted on the seals and this will tend to nullify any possible beneficial effect obtained from the vacuum expansion of other tissue. The invention herein is much more practical. The invention avoids the problems associated with vacuum by using mechanical means to connect to the skin.

The foregoing patents reflect the current state of the art of which the present inventor is aware. Reference to, and discussion of, these patents is intended to aid in discharging Applicant's acknowledged duty of candor in disclosing information that may be relevant to the examination of claims to the present invention. However, it is respectfully submitted that none of the above-indicated patents disclose, teach, suggest, show, or otherwise render obvious, either singly or when considered in combination, the invention described and claimed herein.

### BRIEF SUMMARY OF THE INVENTION

The method and apparatus for treatment of snoring and sleep apnea of this invention provides adhesive patches to exert a predetermined force on two areas of the neck, causing these areas of the neck to expand outward from their normal position. This expansion opens a blockage in the throat of a sleeping person, thereby eliminating snoring and helping some people with obstructive sleep apnea. The force required to expand these two areas of the neck is balanced by pressing on another area of the neck, located generally between the two expanded areas.

The present invention describes how humans have multiple responsive points within a specific area of the neck (hereafter called the “throat stimulation area”) that exhibit the ability to stimulate the throat in such a way as to open the blockage in the air passage of a sleeping person. This eliminates snoring and sleep apnea in some people. To achieve this result, the surface of the skin in at least a portion of the throat stimulation area must be pulled so that the surface of the skin is extended by at least 2.5 mm beyond the normal position. The invention provides an attachment device, such as an adhesive coated patch, that attaches to the skin over at least one responsive point. Pulling on this patch expands the skin and maintains an open-air passage in a sleeping person. The snoring sound is reduced or eliminated and even some people with obstructive sleep apnea can have their breathing passage opened by this invention.

In the preferred embodiment (FIG. 6), adhesive patches are attached to the skin to cover two responsive points along the left side of the trachea and cover two responsive points along the right side of the trachea. The pulling force to expand these areas is generated by pressing on specific locations on the neck found to tolerate pressure without adverse effects. It is therefore an object of the present invention to provide a new and improved method and apparatus for treatment of snoring and sleep apnea.

It is another object of the present invention to provide a new and improved anti-snoring device.

A further object or feature of the present invention is a new and improved anti-snoring device, which is comfortable in use.

An even further object of the present invention is to provide a novel anti-snoring device that expands a portion of the user's neck to facilitate breathing.

The foregoing summary broadly sets out the more important features of the present invention so that the detailed description that follows may be better understood, and so that the present contributions to the art may be better appreciated. There are additional features of the invention that will be described in the detailed description of the preferred embodiments of the invention which will form the subject matter of the claims appended hereto.

Accordingly, before explaining the preferred embodiment of the disclosure in detail, it is to be understood that the disclosure is not limited in its application to the details of the construction and the arrangements set forth in the following description or illustrated in the drawings. The inventive apparatus described herein is capable of other embodiments and of being practiced and carried out in various ways.

Also, it is to be understood that the terminology and phraseology employed herein are for descriptive purposes only, and not limitation. Where specific dimensional and material specifications have been included or omitted from the specification or the claims, or both, it is to be understood that the same are not to be incorporated into the appended claims.

As such, those skilled in the art will appreciate that the conception, upon which this disclosure is based may readily be used as a basis for designing other structures, methods, and systems for carrying out the several purposes of the present invention. It is important, therefore, that the claims are regarded as including such equivalent constructions as far as they do not depart from the spirit and scope of the present invention. Rather, the fundamental aspects of the invention, along with the various features and structures that characterize the invention, are pointed out with particularity in the claims annexed to and forming a part of this disclosure. For a better understanding of the present invention, its advantages and the specific objects attained by its uses, reference should be made to the accompanying drawings and descriptive matter in which there are illustrated the preferred embodiment.

### BRIEF DESCRIPTION OF THE SEVERAL VIEWS OF THE DRAWINGS

The invention will be better understood and objects other than those set forth above will become apparent when consideration is given to the following detailed description thereof. Such description makes reference to the annexed drawings wherein:

FIG. 1 is a diagrammatic perspective view of a human to illustrate the portion of the neck containing the throat stimulation area and responsive points;

FIG. 2 is a diagrammatic perspective view of a human wearing a preferred embodiment of the inventive anti-snoring device, namely a dual adhesive patch with a bow type elastic connector;

FIG. 3 is a perspective view of the bow type elastic connector depicted in FIG. 2;

FIG. 4 is a plan view of one of the adhesive patches depicted in FIGS. 2 and 3;

FIG. 5 is a side view in elevation of the adhesive patch depicted in FIG. 4 showing its coupling relationship to the elastic connector device depicted in FIG. 3;

FIG. 6 is a diagrammatic perspective view showing a human wearing the preferred embodiment of the anti-snoring device;

FIG. 7 is a cross-sectional side view in elevation of the preferred embodiment of the inventive anti-snoring device which utilizes a central pad; and

FIG. 8 is a cross-sectional side view of an alternative embodiment of the anti-snoring device which utilizes a contoured connector.

### DETAILED DESCRIPTION OF THE INVENTION

The instant application is a continuation-in-part of U.S. Utility patent application Ser. No. 11/102,314, filed Apr. 8, 2005, which is a continuation-in-part of U.S. Utility patent application Ser. No. 10/866,058, filed Jun. 10, 2004 (Jun. 10, 2004), each of which application is incorporated in its entirety by reference herein.

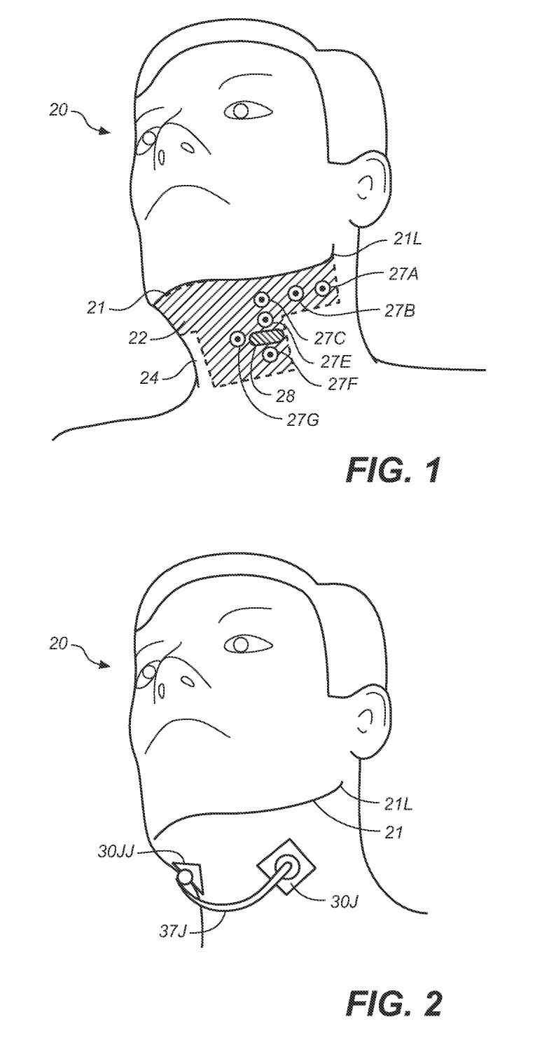

Referring now to FIGS. 1 through 8, wherein like reference numerals refer to like components in the various views, there is illustrated therein a new and improved anti-snoring device.

FIG. 1 shows the head and neck area of a human 20, wherein the lower edge of the jawbone 21 (hereafter called the “jawbone line”), and the left end of the jawbone is 21L are immediately superior to the neck 24. There is a “T” shaped area 22 below the jawbone 21 that will be termed the “throat stimulation area”. Within the throat stimulation area there are several points collectively designated as 27 that will be called “responsive points.” FIG. 1 illustrates responsive points 27A, 27 B, 27C, 27E and 27F, which are on the left side of the neck. Each of these responsive points has a comparable and corresponding point on the right side of the neck, but responsive points on the right side are not illustrated here for simplicity. As noted, responsive point 27G is at the center of the neck (anterior cervical midline), so there are a total of eleven responsive points (including five points on the right not illustrated). Responsive points 27A, 27B and 27C are each located about 1.5 cm below the jawbone line 21. Responsive point 27E is located proximate the left edge of the trachea (windpipe) and about 3 cm below the jawbone line 21. Responsive point 27E is easily located because it is generally the portion of the neck where the carotid artery produces a strong pulse. Since this is the most important of the responsive points, it deserves to have a name and will hereafter be referred to as either responsive point 27E or as the “carotid artery pulse point.” Recall that there are actually two such points, left and right, but the instant specification employs the convention of referring only to the left side for simplicity. FIG. 1 also shows an area 28 that will be discussed later.

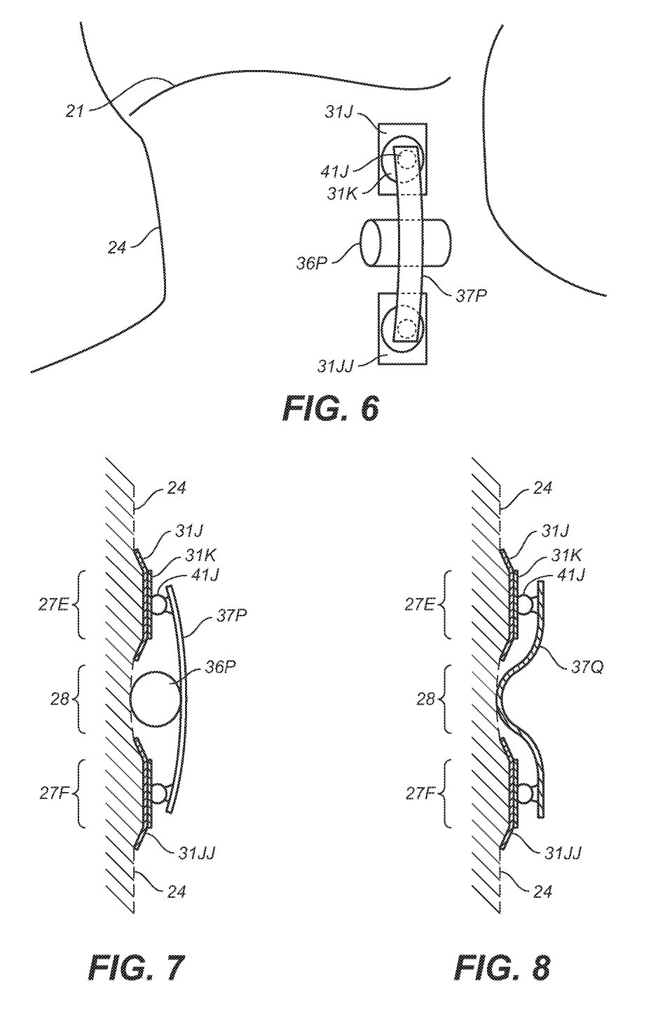

Responsive point 27F is also proximate the left edge of the trachea. On the test subject, responsive point 27F is about 4 cm below the carotid artery pulse point 27E. Responsive point 27E is the lowest of the responsive points and may be termed the “lower neck responsive point.” Finally, and as noted previously, responsive point 27G is located on the anterior cervical midline, or roughly the center of the trachea. Not all the responsive points work the same way and these differences will be discussed in detail below. Responsive points 27E (left and right) are believed to be the most important of the eleven responsive points. Responsive point 27F (left and right side) are also important, but should be used in conjunction with responsive points 27E. The preferred embodiment (FIG. 6) expands both the carotid artery pulse point 27 E and the lower neck responsive point 27F simultaneously.

Throat stimulation area 22 in FIG. 1 encompasses all the responsive points. This throat stimulation area is “T” shaped and the arms of the “T” extend right and left from the anterior cervical midline to the left end of the mandible (jawbone) 22L and the right end of the jawbone (not shown). This top portion (both arms) of the “T” extends about 3 cm below the jawbone line 21. The stem of the “T” (i.e., the vertical portion) extends inferiorly from the lower border of the arms portion to below the superior border of the trachea and approximately 2 cm to the right and left sides of the trachea. The lowest edge of the T-shaped throat stimulation area is approximately 9 cm below the jaw line.

An experiment was performed using a flat piece of plastic 21 square centimeters in area. The plastic was adhered to a subject's neck using double coated tape and covered responsive points 27A, 27B, 27C and 27E. The subject then relaxed muscles in his jaw and neck. This caused the subject's breathing passage to close because the subject suffers from sleep apnea and relaxing the neck muscles simulated the sleeping condition. Very laborious breathing could partly open the breathing passage and caused a snoring sound. It was found that pulling on the plastic immediately opened the blocked air passage. In fact, it was nearly impossible to make a snoring sound as long as there was a pulling force on the plastic and as long as the jaw was positioned with the lower teeth slightly separated from the upper teeth.

The amount of force required to stop the simulated snoring depended on whether the head was tilted forward or back and also depended on the degree of opening between the lower and upper jaw. The simulated snoring sound stopped for a specific head position when the pulling force reached 0.25 Newton (about 25 gram force). This is equivalent to lowering the external pressure on the area covered by the patch by about 120 Pascal (about 12 mm of water). This 120 Pascal pressure reduction is the exact opposite of the 120 Pascal pressure increase sometimes used by continuous positive air pressure (CPAP) machines to open a blocked air passage. This pressure similarity might not be a coincidence since the internal pressure exerted by continuous positive air pressure machines also expands the portion of the neck covered by the patch. It is possible that some of the benefit of CPAP machines is the result of expanding this area. In any case, it appears that in some people, exerting a pulling force on the skin of the neck in the above-described throat stimulation area has a similar beneficial effect to raising the internal pressure on the entire respiratory system.

Not all snoring and sleep apnea sufferers have the same cause of throat blockage. For example, the shape of tonsils, the tongue and the soft pallet differ. There may be additional responsive points within the throat stimulation area that can be found experimentally by those having skill in the art. Therefore, the teaching here is that there are multiple responsive points within the throat stimulation area that can reduce snoring when the skin over one or more responsive point is pulled to expand (lift) the skin surface by at least 2.5 mm beyond its rest position when the tissues in the region are in a generally relaxed state.

FIGS. 2 through 5, taken together, show a single embodiment previously indicated as the preferred embodiment in the progeny application, U.S. patent application Ser. No. 11/102,314, Publication Number 20050274387, titled: “Method and Apparatus for Treatment of Snoring and Sleep Apnea. FIG. 2 shows a human, 20, with two adhesive anti-snoring patches, 30J and 30JJ. These two patches are connected by an elastic connector, 37J, called variously a “bow connector” or an “elastic connector in compression.” The bow connector exerts a separating force on 30J and 30JJ thereby urging them apart and provides the required pulling force on each patch. The patches 30J and 30JJ are illustrated as being placed over previously described carotid artery pulse points 27E (left and right). When this placement is used, successful results have been obtained when the bow connector has a pulling force of 0.36 Newton (37 gram force).

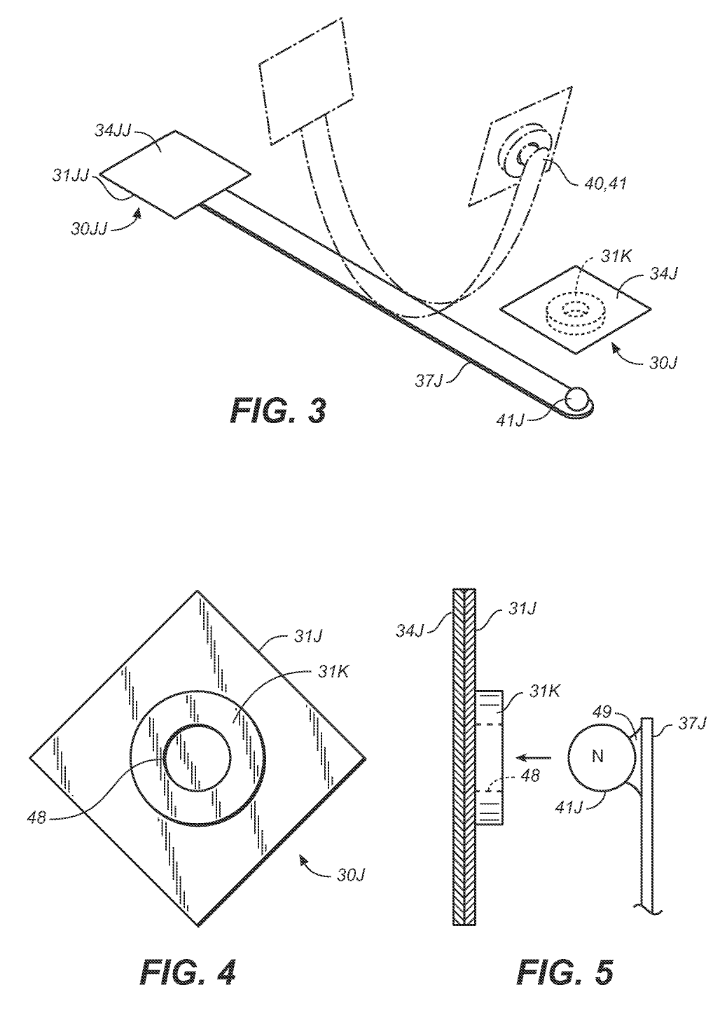

FIG. 3 illustrates the flexibility of the bow connector, 37J. The solid line drawing shows bow connector 37J when no force is being exerted. However, bow connector 37J is fabricated from a resilient, springy material and can be elastically bent (compressed) into the configuration shown by the dashed line drawing in FIG. 3. A strip of either 0.75 mm or 0.62 mm thick polycarbonate plastic, when used for bow connector, 37J, has provided satisfactory performance both structurally and functionally.

FIGS. 3, 4, and 5 illustrate one method of attaching (coupling) the bow connector 37J with patches 30J and 30 JJ. It is desirable that the patches be swivelingly attached so that it can assume different angles relative to the bow connector. This enables it to be adapted to people of different sizes. This calls for a coupling means that has the characteristic of a ball joint. Another desirable characteristic is the ability to easily attach and remove the patches from the flexible bow connector.

The solution to the above-described coupling problem is described in U.S. Pat. No. 6,919,787, to Macken, the inventor herein, and titled “Method and Apparatus for Magnetic Coupling,” which patent is incorporated in its entirety by reference herein. Briefly, FIG. 5 shows a spherical magnet 41J attached to bow connector 37J by welding or an adhesive 49. The letter “N” represents the north pole of the magnet. This implies that the magnetic axis is pointing out of the page. This magnetic orientation means that the spherical magnet 41J will be drawn towards a hole 48 in the annular steel disk 31K. The diameter of hole 48 is optimally slightly smaller than the diameter of the spherical magnet. When spherical magnet 41J contacts the hole 48, it forms a magnetic coupling with the ball joint and includes the release properties previously described.

It is not necessary to use this coupling means to attach patches 30J and 30JJ to the bow coupler 37J. In fact, any elastic means in compression, such as a spring, can be used to supply the required force to exert a force urging patches 30J and 30JJ away from one another.

In one test, the size of bow connector, 37F, was adjusted so that it exerted a 0.33 Newton pulling force (about 34 gram force) on patches 30J and 30JJ. This force was sufficient to open the blocked air passage for a person who suffers from obstructive sleep apnea. Less force may be required for a person with only a snoring problem.

FIGS. 4 and 5, taken together, show details of the construction of patches 30J and 30JJ. The relative thicknesses of components in FIG. 5 are exaggerated for clarity. In this embodiment, annular steel disk 31K is disposed on one side 31J of a piece of tape, which includes a support member or carrier side 31J and adhesive side 34J. Similarly, 31JJ and 34JJ are a corresponding piece of tape for the attachment to other end of the bow connector. Annular disk 31K is another type of support member for the adhesive 34J. In the this embodiment, 31K is an annular steel disk with a hole 48, though it will be appreciated that it could have a number of suitable shapes, as long as it provides hole 48 for supporting of spherical magnetic 41J. The annular steel disk 31K provides the desired stiffness and area so that the experimentally determined optimum area of skin is expanded by the pulling action of the connector. The adhesive could have been applied directly to disk 31K, but it has been experimentally determined that the best adhesion and comfort occurs when the support for the adhesive has both a stiff component 31K and a flexible component (the portion of 31J that extends beyond the edge of 31K).

Other shapes and materials (such as plastic or rubber) can be used for the support member 31 of the adhesive. The reason for the use of steel in this embodiment is to make easy attachment and removal using magnets. Also the use of tape simplifies construction, but it is desirable to cover the adhesive 34J with a release liner prior to usage. A peel-off release liner can be provided and is desirable for storage of a adhesive anti-snoring device prior to use. Therefore, FIG. 5 illustrates the device after the release liner has been removed.

In experiments, 31J and 34J together were a piece of tape, 25 mm×25 mm×0.8 mm thick. Ferromagnetic material 31K was a 0.75 mm thick steel washer, 16 mm outer diameter and 5.6 mm inner diameter. This steel washer was attached to tape 31J. The spherical magnet 41J was 6.35 mm (¼ inch) in diameter and made from the rare earth magnetic material known as NdFeB. The patches 30J and 30JJ were attached to the test subject as shown in FIG. 2. The diamond orientation of the square patches was used because this minimized skin irritation. The patches were applied over carotid artery pulse points 27E previously described in FIG. 1. The bow coupler 37J was then attached using the magnets previously described.

If the test subject normally sleeps with his jaw completely closed (teeth contacting), and if the blockage is strong, then it is advisable for the subject to also wear a commercially available dental spacer to slightly separate the teeth. This is a plastic device that fits over the front two teeth and is usually used to prevent grinding of the teeth. This device is completely different from oral anti-snoring devices that cause the jaw to jut forward unnaturally. Those oral anti-snoring devices are uncomfortable, and can shift the position of teeth.

The preferred embodiment is depicted in FIGS. 6, 7 and 8. However, preliminarily we refer back to FIG. 1 where there is shown an area designated 28. Area 28 is unique because it lies between carotid artery pulse point 27E and responsive point 27F, and most importantly, it is possible to exert pressure on area 28 without adverse effects. Exerting pressure on any responsive points results in blocking the air passage of a sleeping human with a tendency to snoring. This is the opposite of the desired result of opening the air passage. In fact, exerting pressure on most other parts of the throat stimulation area (22 in FIG. 1), also restricts the air passage. Prolonged pressure on portions of the neck can also result in reduced blood flow to the head or possible nerve damage. Therefore, area 28 has a unique combination of location and pressure tolerance. Area 28 will be named the “preferred pressure area.”

FIG. 6 shows a perspective view of the preferred embodiment. Adhesive patch 31J is attached to the neck over carotid artery pulse point 27E (FIG. 1) and adhesive patch 31JJ is attached to the neck over the lower neck responsive point 27F approximately 4 cm below carotid artery pulse point 27E. A soft pad 36P is between the two patches and exerts pressure on the preferred pressure area 28.

Many of the components used in FIGS. 6, 7 and 8 are the same or similar to the components previously explained in FIGS. 3, 4 and 5. The previously explained components include: adhesive anti-snoring patches 30J and 30JJ; spherical magnet 41J; steel disk 31K, and adhesive tape 31J and 31JJ. The connector 37P in FIGS. 6 and 7 is similar to connection 37J in FIGS. 3 and 5, except that 37P is shorter than 37J and not as flexible as 37J. In fact, connector 37P need not be flexible, though a measure of flexibility is desirable.

The key difference between the preferred embodiment shown in FIGS. 6 to 8 and the embodiments shown in the parent application and the progeny cited as a priority documents herein, is that the preferred embodiment herein exerts “pushing force” pressure on the preferred pressure area 28 in FIG. 1 to counteract the pulling force exerted on at least the carotid artery pulse point 27E and preferably exerts a pulling force on both 27E and 27F. In FIGS. 6 and 7, this pushing force is exerted on preferred pressure area 28 by soft pad 36P. This can be seen in FIG. 7 which is a cross-sectional view of the anti snoring device and the neck. The neck skin is designated 24. The adhesive tape is 31J and 31JJ. It can be seen that the neck skin is pulled above its normal level by the two patches with tape 31J and 31JJ. The brackets on the left of FIG. 7 show that the skin is pulled up over carotid artery pulse point 27E and responsive point 27F.

The soft pad 36P makes contact with the skin over preferred pressure area 28. Soft pad 36P exerts sufficient pressure on preferred pressure area 28 to counteract the pulling force exerted on pads 30J and 30JJ.

The coupler 37P transfers the force between pad 36P and patches designated with tape 31J and 31K. In FIG. 7 coupler 37P is shown slightly bent indicating that it is slightly flexible. However, there are many ways of constructing the coupler and flexibility is not a requirement. Also FIG. 7 shows the use of spherical magnets 41J. These are illustrated because they were previously described. However, the magnets represent any attachment means. For example, the coupler and patch may be a single unit molded out of plastic. Furthermore, it is not necessary to use adhesive tape. As an alternative, an adhesive could be applied directly to a plastic or rubber surface and eliminate the adhesive tape. The essential feature is that adhesive surfaces are at each end of the anti-snoring device and a means for exerting pressure on the neck is contained between the two adhesive areas.

FIG. 8 shows a slight modification where pad 36P has been replaced by a contoured connector 37Q. This connector makes contact with the skin and presses on the preferred pressure area 28 to generate the same pushing force as pad 36Q in FIGS. 6 and 7. To generate this predetermined force, connector 37Q should be flexible and contoured in a slightly different shape than shown in FIG. 8. Bending coupler 37Q into the shape shown in FIG. 8 generates the predetermined force against the neck at preferred pressure area 28,

There are infinite variations of possible designs that accomplish the goal of pulling on the skin over the carotid artery pulse point 27E and pushing on the preferred pressure area 28. It is not necessary to utilize the second responsive point over 27F. Expanding the skin over responsive area 27E is by far the most important objective. Therefore, much of the beneficial effects of anti snoring and anti sleep apnea would be achieved without also pulling on the skin over 27F. For example, the coupler 37P or 37Q could be extended and attach to the skin over the collar bone and still obtain most (but not all) the anti snoring and anti sleep apnea benefits.

Therefore, the key elements of the invention are that an elongated anti snoring device has adhesive areas at each end of the device and a central region of the anti-snoring device is designed to exert pressure. The shape and material of the anti-snoring device is such that in use an adhesive pulls on a portion of the neck designated the carotid artery pulse point (27E) and a portion of the anti snoring device presses on a portion of the neck designated the preferred pressure area 28, such that a blocked air passage in a sleeping human is opened, thereby reducing snoring and possibly helping sleep apnea. The preferred usage has the second adhesive surface attached to a portion of the neck designated the “lower responsive point” 27F.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting the scope of the invention, which is defined by the appended claims.

What is claimed as invention is:

1. An anti-snoring apparatus for attachment to a user's throat stimulation area, comprising:
at least one skin attachment means; and
pressure means for exerting pressure against skin; and
coupling means for connecting said skin attachment means to said pressure means such that when said skin attachment means is attached to an area of a user's neck proximate the carotid artery pulse point, and when said pressure means exerts pressure against the lower neck responsive point of the user's neck, said coupling means transfers a force from said pressure means to said attachment means, pulling on said attachment means and causing an expansion of said artery pulse area thereby causing a reduction in snoring in the user.

2. The anti-snoring apparatus of claim 1, wherein said skin attachment means includes an adhesive coated support member.

3. The anti-snoring apparatus of claim 1, wherein said pressure means includes an elastic connector in compression and attached to two of said skin attachment means.

4. The anti-snoring device apparatus of claim 3 wherein said elastic connector is a bow connector and said skin attachment means includes an adhesive coated support member.

5. The anti-snoring apparatus of claim 4, including two coupling means, and wherein said coupling means comprises:
a patch having a first side and a second side, wherein said adhesive coated support member is disposed on said first side;
a ferromagnetic ball receiver disposed on said second side; and
a spherical magnet disposed on said pressure means for partial insertion into, and cradling by, said ball receiver, so as to form a ball joint.

6. The anti-snoring apparatus of claim 5, wherein said ferromagnetic ball receiver has a hole with a diameter slightly smaller than the diameter of said spherical magnet, such that when said spherical magnet contacts the hole in said receiver, it forms a magnetic coupling with the ball joint.

7. The anti-snoring apparatus of claim 3, including first and second skin attachment means, and further including a soft pad adapted for placement between said first and second skin attachment means and on a preferred pressure area when in use, such that said elastic connector exerts a pushing force on said preferred pressure area.

8. The anti-snoring apparatus of claim 3, including first and second skin attachment means, wherein said elastic connector is placed between said first and second skin attachment means when in use, and wherein said elastic connector is contoured so as to exert a pushing force on a preferred pressure area of the user when said first and second skin attachment means are disposed on the skin of the user on the throat stimulation area.
